(19) United States
(12) Patent Application Publication
(10) Pub. No.: US 2024/0083933 A1
(43) Pub. Date: Mar. 14, 2024

(54) DISACCHARIDES FOR TREATING BONE DISEASES

(71) Applicants: UNIVERSITE DE PICARDIE JULES VERNE, Amiens (FR); CENTRE HOSPITALIER UNIVERSITAIRE D'AMIENS-PICARDIE, Amiens (FR); INSERM (INSTITUT NATIONAL DE LA SANTÉ ET DE LA RECHERCHE MÉDICALE), Paris (FR); CENTRE NATIONAL DE LA RECHERCHE SCIENTIFIQUE, Paris (FR)

(72) Inventors: Jérôme AUSSEIL, Toulouse (FR); José KOVENSKY, Paris (FR); Sylvestre TOUMIEUX, Amiens (FR); Eric TRECHEREL, Amiens (FR); Christophe DUSSOUY, Nantes (FR)

(21) Appl. No.: 18/259,368

(22) PCT Filed: Dec. 20, 2021
(86) PCT No.: PCT/FR2021/052408

§ 371 Date: Jun. 26, 2023

(30) Foreign Application Priority Data
Dec. 30, 2020 (FR) FR2014257

(51) Int. Cl. C07H 5/06; A61K 9/00; A61K 47/02; A61P 19/08

(52) U.S. Cl. CPC C07H 5/06; A61K 9/0024; A61K 47/02; A61P 19/08

(57) ABSTRACT

Disclosed is a pharmaceutical composition that contains at least one compound with the following formula (I) as active ingredient:
in which R1 is chosen from H, SO3−, R2 is chosen from H and COCH3, R3 is chosen from H,

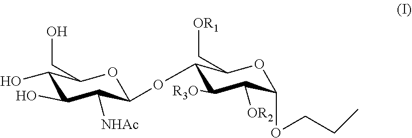

COCH3, benzyl, SO3− and pharmaceutically acceptable salts of the compounds—with the exception of the compound of formula (I) in which R1=SO3− and R2=COCH3 and R3=H and of the sodium salt of the compound—and at least one pharmaceutically acceptable excipient. Also disclosed are an associated kit including a biocompatible medium and methods for treatment.

### TECHNICAL FIELD

The present invention relates to disaccharide-type compounds which are capable of increasing the calcium production of preosteoblastic cells which are therefore useful for the treatment of certain bone diseases.

### PRIOR ART

Bone fractures are frequent traumas and complications of bone consolidation, despite the operation performed by an orthopedist, constitute a medical problem. Indeed, bone consolidation is not a constant phenomenon, there are indeed 10% of cases of fracture that will lead to a non-bone consolidation, and this in the absence of particular risk factor. In contrast, in the case of a technical error (inadequate fastening, rough control of the rotation, in particular in diaphyseal fractures of the long bones), the risk of non-consolidation is estimated at 50%. Between these two extremes, the risk of non-consolidation depends on the presence of local and general factors and can reach 30%. Among these factors, osteoporosis, type I diabetes, alcohol . . . . The fracture related to osteoporosis affects 50% of women and 20% of men over 50 years of age. It is characterized by a deterioration in the quality and the quantity of the bone, which markedly increases the risk of having fractures.

The current reference management of bone defects is autograft. The bone part is harvested either from the cancellous area, or from the cortical area or even the cortico-cancellous area. It allows the addition of the mineral fraction, the protein fraction with its collagenous and non-collagenous matrix proteins. This cell pool is sufficient for the smooth running of the osteogenesis process in the filling site.

The revascularization of a cancellous autograft in the cortical area takes about fifteen days. Then, depending on the species, a delay of one to six months is necessary for its complete integration. This graft has the advantage of being osteogenic, osteoinductive, osteoconductive. However, this technique has significant limitations, including a painful and limited access to the graft site, the large volumes to be harvested by the surgeon as well as the morbidity at the harvest site. It can result in transient lameness of variable intensity and duration. Since the amount of autologous graft is limited, the surgeon can then graft either allogeneic tissue or bone substitute materials. These materials are osteoconductive and are only used as a passive support for bone repair. However, research has been directed towards new solutions, such as BMPs (bone morphogenic proteins) or the percutaneous injection of autologous concentrated bone marrow. The latter consists in introducing mesenchymal stem cells which are at the origin of osteocompetent cells. This technique is accompanied by a minor morbidity while retaining a certain number of the properties of osteoinduction which are dependent on the concentration of stem cells. This solution remains restrictive due to the harvesting step.

BMPs, in turn, are introduced on biocompatible implantable supports. These are osteoinductive endogenous glycoproteins generally released by acidification of the bone matrix (resorption), or during a fracture. They will induce the local recruitment of mesenchymal cells and allow the triggering of the biological cascade leading to the bone formation. They are also synthesized by synthetic biology and marketed as an active ingredient in the field of bone consolidation, under the names of Osigraft® (BMP7), Inductos® and Infuse® Bone Graft (BMP2) in combination with a surgical treatment. These drugs are in the form of a solution to be reconstituted (active ingredient+solvent), then to be introduced on a collagen support (type I bovine collagen). This “soaked” matrix is positioned, during a fracture consolidation operation, by a surgeon on the fractured bone before closing the wound. Marketing authorizations could have been obtained thanks to randomized multicenter studies in humans whose analysis allows better assessing the real effectiveness and the indications of this type of product. The main studies have focused on open fractures of nailed legs, pseudoarthrosis of the long bones, spinal arthrodesis. Studies have also been carried out on osteonecrosis of the femoral head and the thickening of the maxillary sinuses without these allowing obtaining official authorizations. Due to their adverse effects as well as the absence of a good practice for manufacturing the support, the production of BMP2 and BMP7 was stopped in 2016 in Europe.

### Technical Problems to be Solved

A problem which the present invention proposes to solve is to propose disaccharide type compounds which can be used in the treatment of diseases related to a decrease in the bone density and/or in the surgical methods for treating bone defects or bone fractures.

In particular, another problem which the present invention proposes to solve is to propose disaccharide type compounds which are osteoinductive, that is to say they are capable of inducing the cascade of biological mechanisms leading to the formation of the bone.

Another aim of the present invention is to propose compounds as aforementioned which allow a faster regeneration of the bone.

Another aim of the present invention is to propose compounds as aforementioned which prevent the heterotopic formation of the bone, that is to say the formation of bone instead of another tissue.

Another aim of the present invention is to propose a kit allowing the treatment of a bone defect.

### BRIEF DESCRIPTION OF THE INVENTION

The present invention relates to a pharmaceutical composition which, characteristically, contains at least one compound of formula (I) as active ingredient

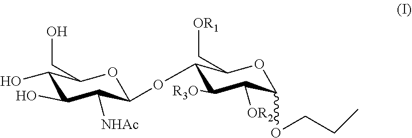

wherein R4 is chosen from H, SO₃⁻, R₂ is chosen from H and COCH₃, R₃ is chosen from H, COCH₃, benzyl, SO₃⁻ and the pharmaceutically acceptable salts of these compounds—with the exception of the compound of formula (I) wherein R₁═SO₃⁻ and R₂═COCH₃ and R₃═H and of the sodium salt of this compound, and with the exception of the compound of formula (I) wherein R₁═R₃═H and R₂═COCH₃—and at least one pharmaceutically acceptable excipient.

Such a composition has proven to be likely to increase the calcium production in preosteoblastic cells and in human osteoblastic cells. The document WO 2009098400 describes the compound with the following formula:

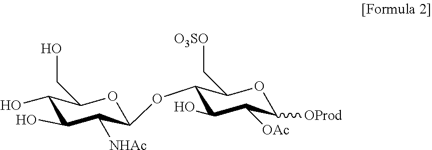

In this document, the aforementioned compound is used as an insecticide. This document also indicates that the aforementioned compound can be used as a drug due to its activity of inhibiting chitinases, which are known to be involved in the mechanisms of allergy and asthma.

The publication entitled “Strong aphicidal activity of GlcNAc(β->4)Glc disaccharides: synthesis, physiological effects and chitinase inhibition” written by C. Dussouy et al and published in Chemistry a European journal (DOI: 10.1002/chem.201200887) describes the synthesis of certain compounds of the invention and the use thereof as insecticides when they are mixed with a food composition allowing feeding insects. Some of these compounds have an activity of inhibiting chitinases but not all.

### DETAILED DESCRIPTION

As regards the pharmaceutically acceptable salts, they may be sodium, lithium or potassium salts, for example, of the aforementioned compounds.

The pharmaceutical composition of the invention may thus contain at least one pharmaceutically acceptable salt, said salt(s) being chosen independently of each other from the sodium salts, the potassium salts and the lithium salts.

Preferably, the composition contains a sodium salt or several sodium salts.

Advantageously, the composition of the invention includes, as active ingredient, only one or more sodium salts.

Advantageously, the composition of the invention is in liquid form. It can thus be injected or used to impregnate a support.

The excipient is not limited according to the invention, it can be chosen from distilled water, the injectable aqueous solutions of sodium chloride, in particular the aqueous solutions containing 9 g/L of sodium chloride.

The pharmaceutical composition of the invention can be administered by injection, subcutaneously, intravenously, by injection into the bones, orally, mucosally, in particular sublingually or nasally. In particular, it can be used by injection directly into a bone defect.

According to a particular embodiment, the pharmaceutical composition of the invention contains, as active ingredient, a mixture of said compound of formula (I) wherein R₁═H; R₂═H and R₃═COCH₃ and of a second compound of formula (I) wherein R₁═H, R₂═COCH₃ and R₃═H. The inventors have demonstrated the appearance of the first aforementioned compound due to a rearrangement during the solvation of the second compound.

Advantageously, the mixture contains ⅔ by weight of the aforementioned compound of formula I in which R₃═H and ⅓ of the second compound.

The pharmaceutical composition of the invention may also, regardless of its embodiment, comprise a calcium phosphate cement capable of being injected and of being solidified in the body of the patient, in particular in a bone or a bone defect of the patient or in a bone area of lower density.

It is thus possible, by injecting the composition of the invention into a bone defect, to fill this defect and to deliver the compounds of the invention which will induce the formation of bone.

The present invention relates to a kit including a biocompatible support, implantable in the body of a subject and at least one compound with the following formula (I):

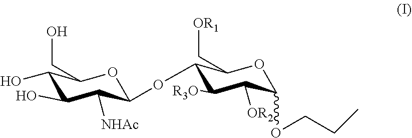

wherein R₁ is chosen from H, SO₃⁻, R₂ is chosen from H and COCH₃, R₃ is chosen from H, COCH₃, benzyl, SO₃⁻ and the pharmaceutically acceptable salts of these compounds.

The inventors have indeed demonstrated that the compounds of the invention are capable of inducing the production of a bone mass on a support.

The biocompatible support is advantageously a support capable of replacing the bone and/or osteoconductive material (that is to say capable of being covered with bone material (bone).

The aforementioned compound can also be in a liquid pharmaceutical composition whose excipient is an injectable solution of sodium chloride, for example. According to the invention, said biocompatible support is advantageously chosen from porous biocompatible supports, in particular supports comprising or consisting of biodegradable polymer(s), in particular PLA, polyglycolic acid, poly(lactic-co-glycolic acid), collagen, polyglycolide, chitosan, polycaprolactone, supports comprising or consisting of ceramic(s), supports comprising or consisting of calcium phosphate, supports including or consisting of the mineral portion of a bone.

Advantageously, the biocompatible support includes or consists of collagen.

The support can be covered with at least one compound according to the invention or impregnated or covered with a pharmaceutical composition containing the compound defined above with reference to the kit of the invention.

The present invention also relates to a compound with the following formula (I):

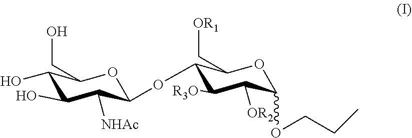

wherein:
a) R₁═R₃═H and R₂═COCH₃
b) R₁═SO₃⁻; R₃═H; R₂═COCH₃
c) R₁═R₂=R₃═H
d) R₁═SO₃⁻; R₂═R₃═H
e) R₁═R₂═H and R₃═COCH₃ and
the pharmaceutically acceptable salts of these compounds for the use thereof alone or in combination of two or more in the treatment of a pathology related to a decrease in bone density, in particular osteoporosis, osteopenia, osteomalacia, osteogenesis imperfecta, diabetoporosis, osteopetrosis, Paget's disease of bone, bone tumors, cancerous bone tumors or for the use thereof alone or in combination of two or more in a surgical method for treating bone defects or bone fractures, in particular pathological fractures. These compounds can be used in combination with the biocompatible support to form particular embodiments of the kit of the invention. According to a particular embodiment, the compounds defined in compounds defined in the aforementioned a) and e) are used in combination for the treatment of the aforementioned pathologies or in a surgical method as aforementioned.

The present invention also relates to a compound with the following formula (I):

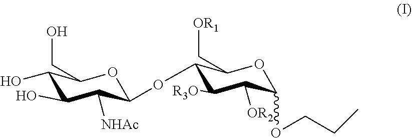

wherein R₁═R₂═H and R₃═COCH₃

Generally, the propoxy substituent of the compound of formula (I) is in position α or β, as indicated by the wavy line in formula (I). In a preferred embodiment of the invention, applicable to all embodiments of the invention, the propoxy substituent of the compound of formula (I) is in position α, as illustrated below.

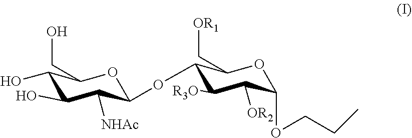

### Definitions

A disease or pathology related to a decrease in bone density designates, within the meaning of the present invention, a pathological state generating, as a symptom, at least locally a decrease in bone density and a pathological state resulting from the decrease in bone density, for example a fracture. Bone tumors and in particular cancerous bone tumors cause a bone fragility, which can be treated by the compounds of the invention.

A pathological fracture is defined as a fracture caused by a decrease in bone density.

A bone defect is, according to the present invention, an area of a bone which has a deficit in bone material; it can be a hole in the bone material or an area where the bone material is less dense.

The bone material covers, within the meaning of the present invention, a solidified or solidifying connective tissue.

The term “treatment” includes the preventive treatment and the curative treatment.

The terms “active ingredients” indicate that the concerned active compound(s) are present in an amount sufficient to obtain a pharmaceutical effect.

### FIGURES

FIG. 1 represents the calcium dosage in the MC3T3-E1 culture medium (in g/mg of protein per well) after 9 days of exposure of these cells to 4 mM of Pi (Pi=Phosphoinositide 3-kinases) (control+Pi) and 4 mM of Pi in the presence of 30 μM of each of the compounds referenced DP2SNA, DP2NA, DP2K and DP2S;

FIG. 2 represents the % increase in the concentration of calcium ions relative to the mineralizing medium alone obtained at D=25 by treatment of the MC3T3-E1 cells with the disaccharides and the sodium salts of these disaccharides DP2 and DP2NA, respectively:

FIG. 3 represents the percentage increase in the concentration of calcium ions relative to the mineralizing medium alone obtained at D=14 by treatment of HOb cells under mineralizing conditions in the presence of DP2 and DP2NA at 15, and 50 μM:

FIG. 4 represents the percentage increase in the concentration of calcium ions relative to the mineralizing medium alone obtained at D=14 by treatment of the HOb cells under mineralizing conditions in the presence of DP2 and DP2NA at 5, 7.5, 10, 15, 30 and 50 μM:

FIG. 5 represents the amount of calcium (in μg) produced by smooth muscle cells in the presence of DP2, the cells were treated under mineralizing conditions in the presence of DP2 5, 7.5, 10, 15, 30 and 50 μM;

FIG. 6 represents the percentage of mineralization of the HOb cells obtained after 14 days of treatment under the mineralizing conditions in the presence of DP2R0 at 30 μM and BMP-2 100 ng/ml or the mixture thereof;

FIG. 7 represents the percentage of mineralization obtained on HOb cells treated for 14 days under mineralizing conditions in the presence of: (7A) DP2R2 at 30 μM and (7B) DP2R2 or DP2′ at 30 μM and (7C) DP2R2 marked at 95% purity (DP2M95 μM);

FIG. 8 represents the enzymatic activity of alkaline phosphatase (U/L/ug of proteins) in MC3T3-E1 and HOb cells, obtained for: (8A) MO3T3-E1 cells at D=18 days in the presence or absence of DP2R0 at 15, 80 and 50 μM compared with BMP-2 at 100 ng/ml (8B) the HOb cells at D=7 days by the mineralizing medium in the presence or absence of DP2R0 at 30 μM compared with BMP-2 and BMP-7 at 100 ng/ml, (8C) the HOb cells at D=7 treated by the mineralizing medium in the presence or absence of DP2R2 at 30 μM compared with BMP-2 and BMP-7 at 100 ng/ml.

FIG. 9 represents the results of the DP2 cytotoxicity tests at 30 μM on HOb cells at D=14 using the MTT test (9A) and using the WST-1 test (9B);

### EXPERIMENTAL PART

Chemical Synthesis of the Compounds of the Invention

The syntheses of the compounds are as described in the publication “Strong aphicidal activity of GIcNAc(β->4)Glc disaccharides: synthesis, physiological effects and chitinase inhibition” written by C. Dussouy et al and published in Chemistry a European journal (DOI: 10.1002/chem.201200887)

Synthesis of the Compound DP2′

The compound DP2′ was extracted from the DP2-DP2′ mixture which is formed when the compound DP2 is dissolved in water. The compound DP2′ has been separated by preparative HPLC chromatography with water as elution solvent.

The inventors have observed that when the compound DP2′ was in water, the delocalization of the acetate group was also present, but with very slow kinetics compared to the DP2→DP2′ reaction. Thus, a solution of DP2′ in water which is left to rest contains, after 44 days, 10 to 15% of DP2. The DP2′ therefore remains alone in the water for a given duration which is quite long. It is therefore possible to test the biological effect of DP2′ in solution in water.

Similarly, the inventors have observed that the kinetics of the DP2→DP2′ reaction is such that it is also possible to test the biological activity of the compound DP2 alone after extraction of the formed compound DP2′. Thus, the two compounds DP2 and DP2′ can coexist or not in water for a given duration.

Moreover, in other solvents, such as methanol, no delocalization of the acetate group has been observed. In methanol, for example, the compound DP2 is therefore alone.

Used References

The following references: DP2, DP2R0, DP2R2, DP2K correspond to the compound n-propyl, 2-O-acetyl-α-D-Glucopyranoside, 4-O-(2-acetamido-2-deoxy-β-D-glucopyranosyl) with the following formula I:

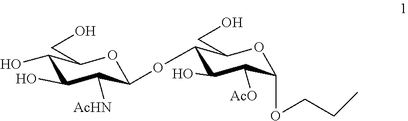

The nomenclatures R0, R2 and K refer to production batches of the DP2 molecule.

The reference DP2′ corresponds to the compound n-propyl, 3-0-acetyl-α-D-Glucopyranoside, 4-O-(2-acetamido-2-deoxy-β-D-glucopyranosyl) with the following formula 1′:

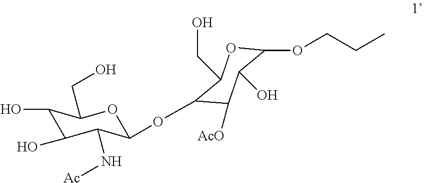 [SMILES: CCCOC1OC(CO)C(OC2OC(CO)C(O)C(O)C2NC(C)=O)C(OC(C)=O)C1O]

The reference DP2S corresponds to the compound of formula (I) wherein R₁═SO₃⁻ and R₂=Ac, such as n-propyl, 2-0-acetyl-6-O-sulfo-α-D-Glucopyranoside, 4-O-(2-acetamido-2-desoxy-β-D-glucopyranosyl), monosodium salt.

The reference DP2NA corresponds to the compound n-propyl, α-D-Glucopyranoside, 4-O-(2-acetamido-2-deoxy-β-D-glucopyranosyl) with the following formula 3:

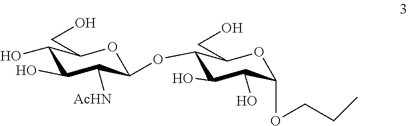

The reference DP2SNA corresponds to the compound n-propyl, 6-O-sulfo-α-D-Glucopyranoside, 4-O-(2-acetamido-2-desoxy-β-D-glucopyranosyl), monosodium salt and with the following formula 4:

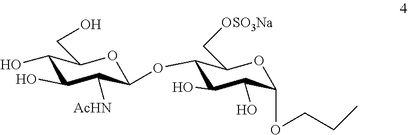

In-Vitro Experiments

Cellular Culture

For all experiments, a mouse pre-osteoblastic cell line (MC3T3-E1, ATCC) and human primary osteoblastic cells (HOb, Promocell) were used. The mouse pre-osteoblastic cells were cultured in 75 cm² flasks, containing 15 mL of α-MEM medium (M4526, SIGMA) and 10% fetal calf serum (FCS), containing 1% Penicillin/Streptomycin antibiotics and 1% glutamine. Similarly, human HOb cells were cultured in 75 cm 2 flasks containing 15 mL of proliferation medium (Human Osteoblast Proliferation Medium, PromoCell). The cells are maintained in the oven at 37° C., with 5% CO₂ and 90% humidity. The medium was changed every three days.

MC3T3-E1 cells were seeded in 6-well plates at 63,700 cells/well to be treated in order to extract RNA or for alkaline phosphatase enzymatic activity assay and in 48-well plates for the calcium assay at 5250 cells/well. The HOb cells were seeded at 182,000 cells/well in 6-well plates for the extraction of RNA or for the alkaline phosphatase enzymatic activity assay; at 15,000 cells/well in 48-well plates for the calcium assay; at 10,000 cells/well in 48-well plates for the MTT test or the WST-1 test and at 100,000 cells/well in 6-well plates for the protein extraction or for the fluorescence.

Cellular Treatment

In order to induce the mineralization, the MC3T3-E1 cells were treated with α-MEM medium containing 5% FCS to which 10 mM of β-glycerophosphate and 50 μg/mL of ascorbic acid are added. The HOb cells were treated with the “Osteoblast mineralization medium” (Promocell). According to the conditions, the DP2NA, DP2R0, DP2R2, DP2′, BMP-2 or BMP-7 were added at different concentrations. After filtration, the cells were treated with the respective media (500 μL per well of the 48-well plate and 2 mL per well of the 6-well plate).

Pro-Calcifying Activity of the Compounds of the Invention

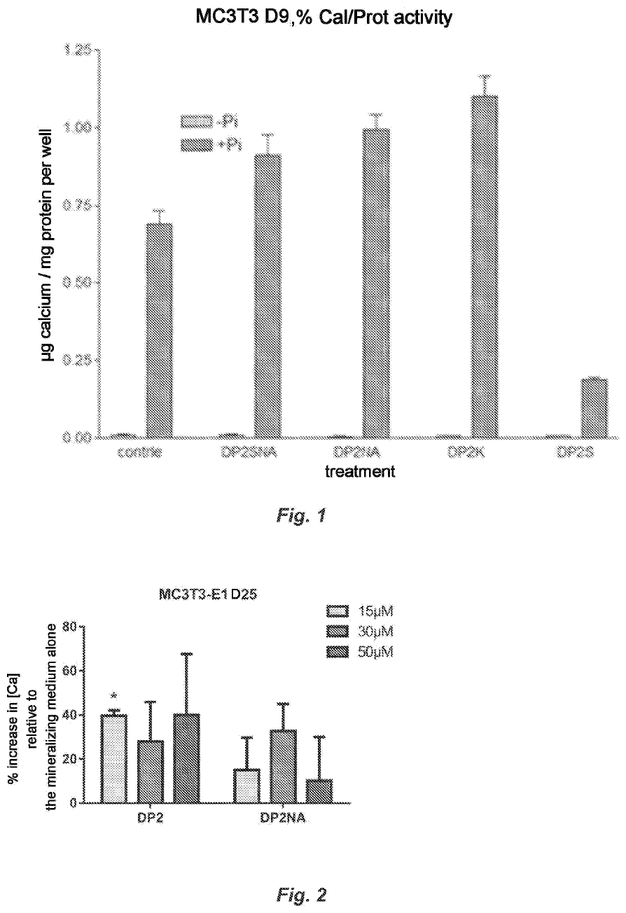

The release of calcium in the culture medium of mouse pre-osteoblastic cells (MC3T3-E1) has been studied. The results are shown in FIG. 1: they clearly show that 3 DP2 (DP2SNA; DP2NA: DP2K) have the ability to increase the production of calcium by the MC3T3-E1 cells unlike DP2S which significantly inhibits the expression of calcium.

Following these results, the pro-calcifying activity on the MC3T3-E1 treated with DP2NA and DP2R0, for 25 days, was studied. An increase in calcification was observed, relative to the mineralizing medium (FIG. 2), in the presence of DP2R0 at 15 μM (40±2%), at 30 μM (28±18%) and at 50 μM (40±28%), and DP2NA at 15 μM (15±14%), at 30 μM (33±12%) and at 50 μM (10±19%).

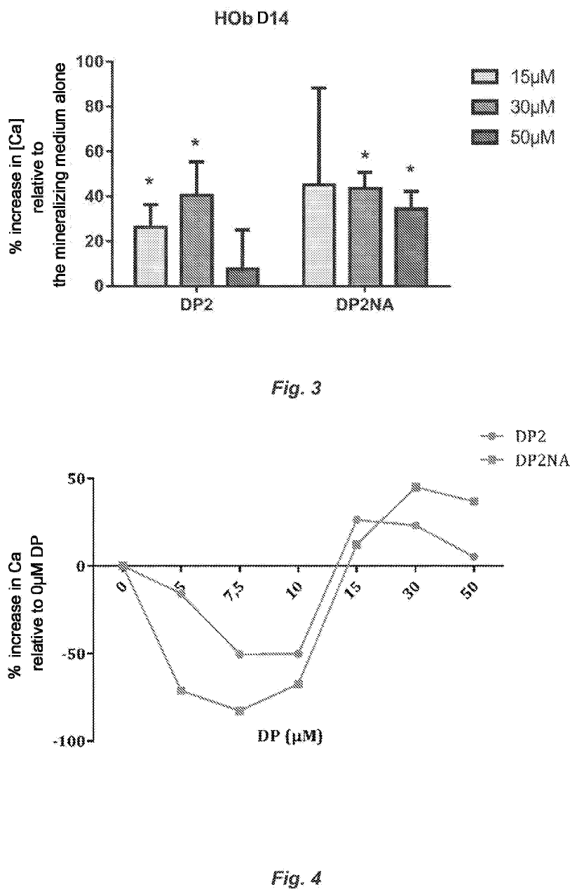

The calcification has also been studied on human osteoblastic cells (Hob, PromoCell, Heidelberg, Germany). They are isolated from femoral trabecular bone tissues. Following a 14-day treatment in a mineralizing medium (PromoCell), on the primary HOb cells, a significant increase in the calcification was observed (see FIG. 3), relative to the mineralizing condition, in the presence of DP2 at 15 μM (26±10%), at 30 μM (40±15%) and DP2NA at 30 μM (44±7%) and at 50 μM (35±8%). An increase observed in the presence of DP2 at 50 μM (8±17%) and DP2NA at 15 μM (45±42 9 6), but which is not significant because of the standard deviation.

Following the treatment of the HOb cells with DP2R0 and DP2NA at lower concentrations (5, 7.5 and 10 μM), a decrease in mineralization of the HOb cells was observed relative to the mineralizing medium alone (FIG. 4). This suggests that low concentrations of DP2 have no action on the mineralization and therefore that at a distance, contrary to what has been described in the case of BMPs, there would be no development of heterotopic bone in the presence of DP2.

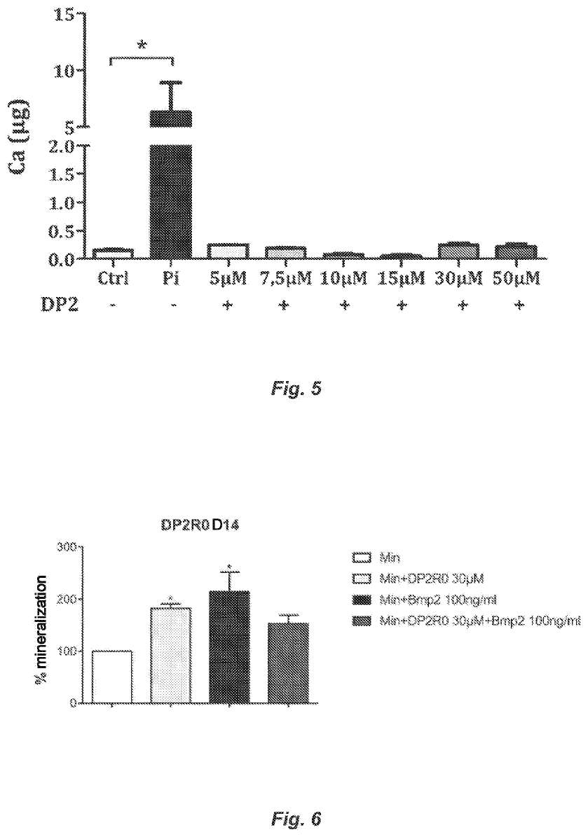

Moreover, the results shown in FIG. 5 show that in non-calcifying control condition, the presence of DP2 has no pro-calcifying action on the smooth muscle cells. This therefore reinforces the hypothesis that DP2 will not induce any calcification on cells other than the osteoblastic cells.

Based on these results, the studies were continued by choosing the DP2R0 as molecule of interest. The results shown in FIG. 6 show that DP2R0 30 μM and BMP-2 100 ng/ml increase the mineralization of 181.573±9.155% and 214.153±37.938% respectively. This indicates that DP2R0 30 μM has an activity of the same order of magnitude as BMP-2 at the concentration defined in the literature (100 ng/ml). The synergistic effect of DP2 with BMP-2 on the calcification has also been studied on human osteoblastic cells. The results visible in FIG. 6, show that DP2R0 and BMP-2 (152.950±16.176%) do not appear to have a synergistic effect on human osteoblastic cells.

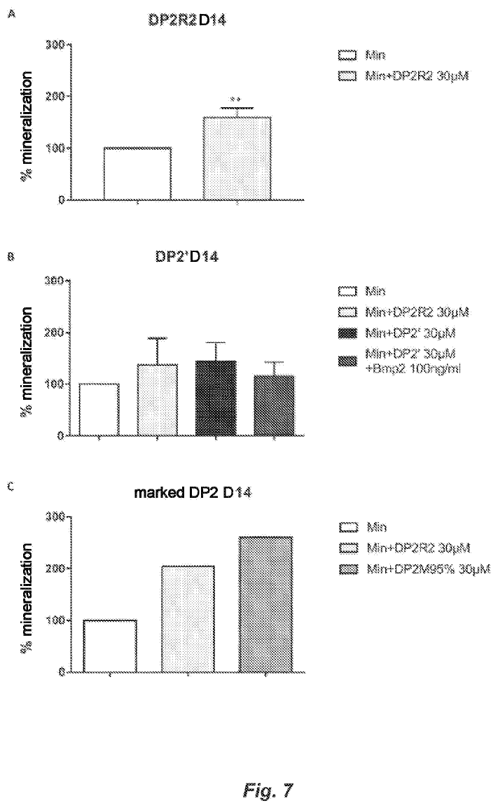

The service provider Roowin supplied a DP2 molecule called DP2R2 (2^{nd} produced batch), hence the name DP2R0 used above to designate the DP2 produced by the LG2A laboratory (UPJV, Amiens). The pro-calcifying activity of this molecule has been studied and the results are shown in FIG. 7A below (DP2R2: 159.03±18.719%).

The chemical analyzes of the aqueous solution of DP2R2 showed that the compound DP2R2 (DP2) reacts with water to give the compound DP2′. There is about ⅔ of DP2R2 for ⅓ of DP2′. The compound DP2′ comes from a migration of the acetate group (R2) on the 30H position. The compound DP2′ could be isolated and synthesized as previously indicated. When the compound DP2′ is mixed with water, the reverse reaction which leads to the formation of DP2R2 is not observed and only the compound DP2′ is present in solution. The pro-calcifying activity of this molecule has also been studied, the results (FIG. 7B) show that DP2′ at 30 μM seems to have an activity of the same order of magnitude as DP2R2 at 30 μM (143.806±36.657% and 137.012±52.452% respectively).

In order to study the hypothesis of the cell penetration of our molecule, DP2R2 has been coupled to a fluorophore: fluorescein. Thus, the pro-calcifying activity has also been tested and the results are shown in FIG. 7C. The mineralization is 204.472% for DP2R2 30 μM and 260.479% for DP2R2 marked at 95% purity (DP2M95 30 μM).

Alkaline Phosphatase (ALP) Enzymatic Activity Assay

ALP is an enzyme synthesized by osteoblasts. It hydrolyses phosphoric esters which inhibit the mineralization by releasing a hydroxyl group and a phosphate. The osteoblasts produce matrix vesicles, reservoirs of alkaline phosphatases and ions, which, when released into the extracellular medium, would initiate the mineralization of the osteoid tissue by promoting the local concentrations of calcium ions and phosphates. Cells have been seeded in 6-well plates at 63700 cells/well for MC3T3-E1 and at 182000 cells/well for HOb. The assay has been performed on a 48-well plate after 13 days of treatment for MC3T3-Et cells and 7 days of treatment for the HOb cells using the BioVision Alkaline Phosphatase activity colorimetric assay Kit (ALP Assay Buffer, pNPP tablets, Alkaline phosphatase enzyme, Stop solution).

After the PBS rinses, 50 μl of ALP Assay Buffer were added to the wells, the bottom of the wells was scraped and then the 50 μl were taken. A centrifugation for 3 minutes at 13000 G was carried out to remove the insoluble material. In a 96-well plate, 10 μl of each sample to be tested are placed with 70 μl of the ALP Assay buffer and 50 μl of the 5 mM pNPP solution. In parallel, the calibration range was prepared by placing, in the same 96-well plate. The reaction was incubated for 60 min at 25° C., shielded from light. The reaction was stopped by adding 20 μl of the Stop Solution. The optical density was measured at 405 nm.

After the ALP enzymatic activity assay, a protein assay was performed in order to normalize the results of the ALP assay depending on the number of proteins. To assay the proteins, the colorimetric method with the Pierce™ BCA Protein Assay Kit from thermofisher was used. In 5 mL tubes, a standard range from a 2 mg/mL albumin solution was prepared. In a 96-well plate, 5 μl of the standard range and samples to be tested were dispensed. Then 200 μl of the kit reagent was added to the wells. The reaction was incubated for 15 minutes at 56° C. The optical density was measured at 565 nm to determine the amount of proteins.

Alkaline phosphatase is an enzyme that plays an essential role in the bone formation. During osteoblast differentiation, the expression of alkaline phosphatase by osteoblasts increases gradually with time.

For this, the alkaline phosphatase enzymatic activity on MC3T3-E1 and HOb cells was studied.

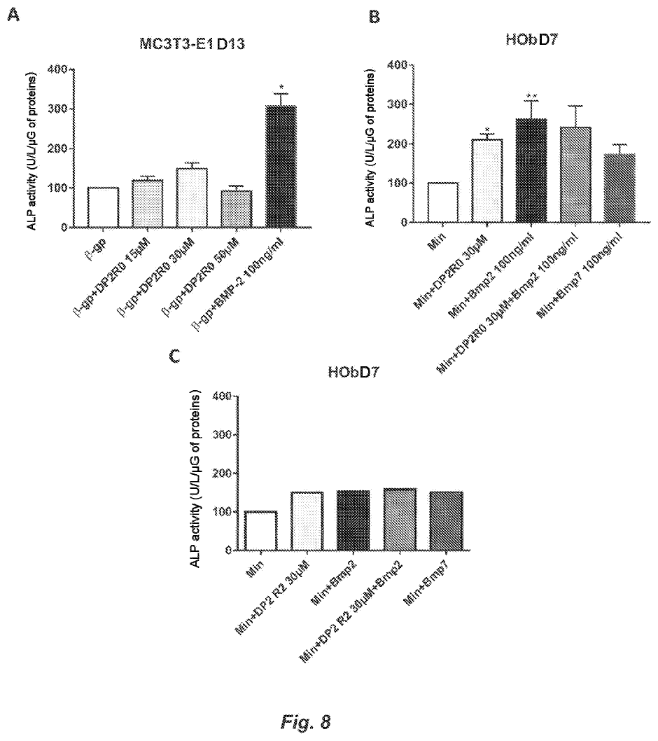

On the MC3T3-E1, 13 days post-treatment, the enzymatic activity is slightly increased in the presence of DP2R0 15 μM (118.907±10.466%) and DP2R0 15 μM (148.833±15.761%) relative to the mineralized medium alone (100%). This increase is more significant in the presence of BMP-2 at 100 ng/ml (307.645±30.440%: FIG. 8A).

More markedly on the H0b, 7 days post-treatment, the enzymatic activity is significantly increased in the presence of DP2R0 30 μM (210.494±14.979%) and BMP-2 100 ng/ml (262.567±46.207%) relative to the mineralized medium alone (100%). Similar to what is observed for bone calcification, the results show that DP2 and BMP-2 do not seem to have a synergistic effect on human osteoblastic cells (FIG. 8B).

The ALP enzymatic activity has also been confirmed in the presence of DP2R2 30 μM (149.749%) relative to the mineralized medium alone (100%) (FIG. 8C).

Cytotoxicity Tests

MTT Test

In order to study the toxicity of DP2, cell viability was determined using the MTT test (3-(4,5-dimethyl-2-thiazolyl)-2,5-diphenyl-2H-tetrazolium bromide). The HOb cells were seeded in 48-well plates (10,000 cells per well), two days later the cells were treated with DP2 30 μM and the MTT test was carried out on D2, D4, 7 and D14 post-treatment.

After washing the cells with DMEM (Dulbecco's Modified Eagle Medium) without phenol red, 250 μl of the diluted MTT solution were added to each well and the plate is incubated at 37° C. with 5% CO₂ for 1 hour. The amount of MTT reduced in formazan is proportional to living cells. The purple formazan crystals were solubilized in 220 μl of DMSO and after stirring to homogenize the coloration, the absorbance was measured at 560 nm (Envision, PerkinElmer). The cytotoxicity was assessed by comparing the cell viability between the control group and the experimental group.

WST-1 Test

The toxicity of DP2 has also been studied by another cell viability test: WST-1 (a tetrazolium salt). The HOb cells were seeded in 48-well plates (10,000 cells per well), two days later, the cells were treated with DP2 30 μM and the WST-1 test was carried out on D2, D4, D7 and D14 post-treatment.

At the end of the treatment, 100 μl of the diluted WST-1 solution (Roche Diagnostics) was added to each well and the plate is incubated at 37° C. with 5% CO2 for 1 h. The cell proliferation induces an increase in the activity of mitochondrial dehydrogenases, which cleaves the tetrazolium salt into formazan. The amount of formazan dye was quantified by measuring the absorbance of dye at 450 nm (Envision, PerkinElmer). The results are expressed as percentage of viability in comparison to the untreated control condition.

Before determining whether our molecule alters the survival of osteoblastic cells, its cytotoxicity was tested on the HOb cells by the MTT and WST-1 tests. The cells were treated with DP2 at 30 μM with the culture medium (Ctrl) or the mineralizing medium (Min) for 2, 4, 7 and 14 days (D).

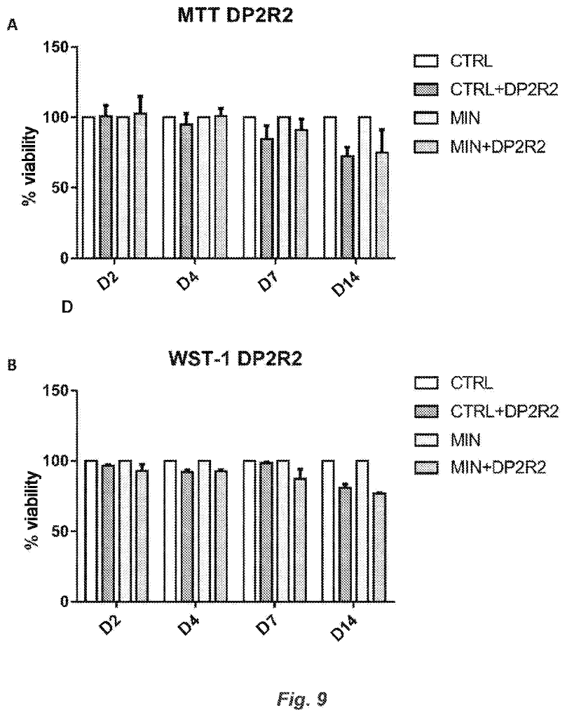

As shown in FIG. 9, there is no significant difference observed between the different treatment conditions for the 4 tested times. A decrease in cell viability is observed on D14 due to osteoblast apoptosis, which correlates with the induced calcification. These results suggest that DP2 at 30 μM have no toxicity to the HOb cells.

In-Vivo Experiments

Animals

Animal housing and the experimental procedures have been carried out in accordance with the European Union directive (2010/63/EU) and validated by the ethics committee (CREMEAP, CEEA N^{o} 96; APAFIS #15464-2018060810566765 v2) from the University of Picardy Jules Verne (UPJV)

The Sprague-Dawley rats are provided by Janvier Labs (Le Genest-Saint-Isle, France) and housed in the PLATAN animal facility of the UPJV. The rats were maintained under a 12-hour day-night cycle, at a temperature of 22±2° C. and 50% humidity. All rats had a free access to water and food pellets, and the Weight changes of the rats were supervised.

Design of the Study

In order to assess the osteoinductive capacity of the disaccharide via the acceleration of the phenomenon of bone reconstruction and to determine the concentration of the optimal DP2 molecule, and the nature of the most adapted support under the physiological conditions of the fracture consolidation operation (inflammatory reaction, cellular activity), 2 bone defects were made in the skull of rats.

Three different types of supports were tested and two different concentrations of DP2. For this, 102 8-week-old male Sprague-Dawley rats were divided into 12 experimental groups such as:
Support 1: Bioactive calcium phosphate bone substitute (MBCP: Biomaltante, Vigneux-de-Bretagne, France)
   Group 1 (S1L1): MBCP support only
   Group 2 (S1L2): MBCP support+DP2 [3 mM]
   Group 3 (S1L3): MBCP support+DP2 [9 mM]
   Group 4 (S1L4): MBCP support+BMP-2
Support 2: bone substitute consisting of the mineral portion of a bone of bovine origin (Bio-oss; Roissy-en-France; France)
   Group 5 (S2L5): Bio-oss support only
   Group 6 (S2L6): Bio-oss Support+DP2 [3 mM]
   Group 7 (S2L7): Bio-oss support+DP2 [9 mM]
   Group 8 (S2L8): Bio-oss support+BMP-2
Support 3: Type I collagen of the bovine Achilles tendon (Sigma)
   Group 9 (S3L9): Collagen support only
   Group 10 (S3L10): Collagen support+DP2 [9 mM]
   Group 11 (S3L11): Collagen support+DP2 [9 mM]
   Group 12 (S3L12): Collagen support+BMP-2

In order to reduce the number of used rats, two bone defects were made per rat; one defect was left blank and was used as an internal control for each rat. Thus, the support-only group is considered the control of the experiment. DP2 and BMP-2 were diluted in sterile saline solution and then mixed with the support and placed in the cranial defect. For the support-only condition, the latter is mixed with sterile saline solution.

Surgery

After being assigned to one of the 12 groups shown above, the rat was anesthetized using IsoVet (isoflurane) in the mask (5% in the induction and 2 to 3% in maintenance dose). After 5 min, a nociception test was performed by pinching the paw or tail of the animal to check the effectiveness of the anesthesia. On the workstation, a shaving and a disinfection with vetedine were then carried out next to the incision.

A local anesthesia was also carried out with 7 mg/kg of 2% lidocaine (20 mg/mL) diluted to 5 mg/ml.

On the workstation, the incision of the skin with a 15 blade cold scalpel along the midline was performed, followed by the detachment of the subcutaneous tissues. Then, the incision along the midline and the lifting and lateral displacement of the periosteum were made. Using a burr, two cranial lesions were performed with 5 mm diameter on each side of the sagittal suture by spacing as much as possible and without touching the superior sagittal sinus. Saline solution was used during the milling steps. The implantation of the support, depending on the group, on the left side was made; the right side, without support, was used as a control to assess the effect of the lesion and the natural repair processes without the presence of support. The periosteum was put back in place and sutured with 7/0 skin suture. The skin was also put back in place and closed with 4/0 skin suture.

The rat was injected with buprenorphine at a dose of 0.05 mg/kg subcutaneously as an analgesic. After awakening, the rat was returned to its conventional housing conditions, by putting 1 rat per cage. The animals were supervised daily and animal welfare monitoring was carried out.

All animals survived for the duration of the study without complications. After 3 months, there were no clinical signs of infection, hematoma or necrosis to defect sites. This indicates that DP2 does not have any in-vivo toxic effect. In addition, all rats showed a homogeneity in bone reconstruction, this was assessed by calculating the values obtained in all empty holes which have been used as an internal control for each rat. A significant increase (the peak) in bone repair is observed between D14 and D63; then there has been a less significant increase, such as a plateau that begins at D63. The results obtained for the 3 supports are summarized in Table 1 below.

| | | TABLES 1 | | | | | | | |
|---|---|---|---|---|---|---|---|---|---|
| | | Results | | | | | | | |
| | | D 14 | | D 29 | | D 63 | | D 91 | |
| Condition | | BMD | BV/TV | BMD | BV/TV | BMD | BV/TV | BMD | BV/TV |
| Support | Treatment | (g/cm3) | (%) | (g/cm3) | (%) | (g/cm3) | (%) | (g/cm3) | (%) |
| MBCP | MBCP only | Reference value | | | | | | | |
| | DP2R2 3 mM | = | = | ↑* | = | ↑* | = | ↑*** | = |
| | DP2R2 9 mM | = | = | ↑* | = | ↑*** | = | ↑* | = |
| | BMP-2 | = | = | ↑* | = | = | = | ↑* | = |
| | Collagen + | ↓*** | N/A | ↓*** | N/A | ↓*** | N/A | ↓*** | N/A |
| | BMP-2 | | | | | | | | |
| Bio-oss | Bio-oss only | Reference value | | | | | | | |
| | DP2R2 3 mM | = | = | = | = | = | = | = | = |
| | DP2R2 9 mM | = | = | = | = | = | = | = | = |
| | BMP-2 | = | = | = | = | = | = | = | = |
| | Collagen + | ↓*** | N/A | ↓*** | N/A | ↓*** | N/A | ↓*** | N/A |
| | BMP-2 | | | | | | | | |
| Collagen | Collagen only | Reference value | | | | | | | |
| | DP2R2 3 mM | ↑* | = | = | = | = | = | = | = |
| | DP2R2 9 mM | = | = | = | = | = | = | = | = |
| | BMP-2 | = | = | = | = | = | = | = | = |

(=: no difference, N/A: not applicable, *p ≤ 0.05, **p ≤ 0.01, ***p ≤ 0.001)

The results in Table 1 show that the 2 MBCP+DP2 groups increase the bone mineralization relative MBCP alone from D29 post-surgery. The 3 Bio-oss groups do not have any effect relative to Bio-oss alone, this was also observed in a model of cranial defect in rabbits (Leventis et al. 2018). This can be explained by a problem of release by the support. Only the collagen+DP2 [3 mM] group increases the bone mineralization on D14.

Interestingly, it was noticed that the MBCP+BMP-2 group forms heterotopic bone over time, this appears to be similar to what is observed as an adverse effect of BMP-2. In addition, the collagen+BMP-2 group does not form a continuous bone repair between the old bone and the newly formed bone, which is not the case with the compound DP2.

1. A pharmaceutical composition comprising at least one compound of formula (I) as active ingredient wherein: 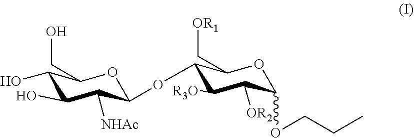
R4 is chosen from H, SO₃⁻,
R2 is chosen from H and COCH₃,
R3 is chosen from H, COCH₃, benzyl, SO₃⁻ and the pharmaceutically acceptable salts of these compounds
with the exception of the compound of formula (I) wherein R₁═SO₃⁻ and R₂═COCH₃ and R₃═H and of the sodium salt of this compound, and with the exception of the compound of formula (I) wherein R₁═R₃═H and R₂═COCH₃
and at least one pharmaceutically acceptable excipient.

2. The pharmaceutical composition according to claim 1, comprising at least one pharmaceutically acceptable salt and wherein said salt(s) are chosen independently of each other from the sodium salts, the potassium salts and the lithium salts.

3. The pharmaceutical composition of claim 1, wherein said excipient is chosen from the injectable solutions of sodium chloride.

4. The pharmaceutical composition according to claim 1, comprising, as active ingredient, a mixture of said compound of formula (I) wherein R₁═H; R₂═H and R₃═COCH₃ and of the compound of formula (I) wherein R₁═H, R₂═COCH₃ and R₃═H.

5. A kit including a biocompatible support, implantable in the body of a subject and at least one compound with the following formula (I): 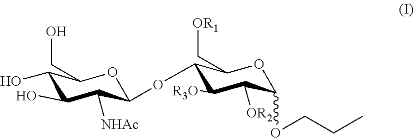
wherein R₁ is chosen from H, SO₃⁻, R₂ is chosen from H and COCH₃, R₃ is chosen from H, COCH₃, benzyl, SO₃⁻ and the pharmaceutically acceptable salts of these compounds.

6. The kit according to claim 5, wherein said biocompatible support is chosen from porous biocompatible supports, supports comprising or consisting of ceramic(s), supports comprising or consisting of calcium phosphate, supports including or consisting of the mineral portion of a bone.

7. A method either for treatment of a pathology generating, as a symptom, at least locally a decrease in bone density, or in a surgical procedure for treating bone defects, or for treating bone fractures, the method comprising administering an effective dose to a patient in need thereof of compounds, either alone or in combination of two or more, with the following formula (I): 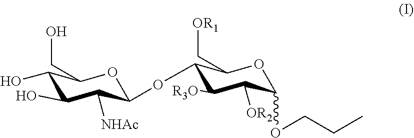
wherein:
a) R₁═R₃═H and R₂═COCH₃
b) R₁═SO₃⁻; R₃═H; R₂═COCH₃
c) R₁═R₂=R₃═H
d) R₁═SO₃⁻; R₂═R₃═H
e) R₁═R₂═H and R₃═COCH₃ and
the pharmaceutically acceptable salts of these compounds.

8. The method of claim 7, wherein the compounds defined in a) and e) are used in combination.

9. A compound with the following formula (I): 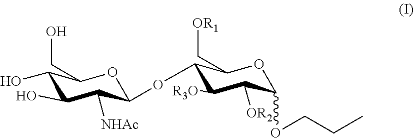
wherein R₁═R₂═H and R₃═COCH₃.

10. The kit of claim 6, wherein the support is a biocompatible support comprising biodegradable polymer(s).

11. the kit of claim 10, wherein the support is selected from the group consisting of: PLA, polyglycolic acid, poly(lactic-co-glycolic acid), collagen, polyglycolide, chitosan, and polycaprolactone.

12. The method of claim 7, performed for treatment of a pathology selected from the group consisting of: osteoporosis, osteopenia, osteomalacia, osteogenesis imperfecta, diabetoporosis, osteopetrosis, Paget's disease of bone, bone tumors, and cancerous bone tumors.

13. The method of claim 7, wherein the method is performed as part of a surgical procedure for treating bone defects, wherein the bone defect is an area of a bone which has a deficiency in bone material.

14. The method of claim 7, wherein the method is performed for treating a pathological bone fracture.

15. The pharmaceutical composition of claim 2, wherein said excipient is chosen from the injectable solutions of sodium chloride.

16. The pharmaceutical composition according to claim 2, comprising, as active ingredient, a mixture of said compound of formula (I) wherein R₁═H; R₂═H and R₃═COCH₃ and of the compound of formula (I) wherein R₁═H, R₂═COCH₃ and R₃═H.

17. The pharmaceutical composition according to claim 3, comprising, as active ingredient, a mixture of said compound of formula (I) wherein R₁═H; R₂═H and R₃═COCH₃ and of the compound of formula (I) wherein R₁═H, R₂═COCH₃ and R₃═H.

18. The pharmaceutical composition according to claim 15, comprising, as active ingredient, a mixture of said compound of formula (I) wherein R₁═H; R₂═H and R₃═COCH₃ and of the compound of formula (I) wherein R₁═H, R₂═COCH₃ and R₃═H.
